(19) United States
(12) Patent Application Publication
(10) Pub. No.: US 2024/0309013 A1
(43) Pub. Date: Sep. 19, 2024

(54) SYNTHESIS OF 2'-(7,7-dimethyl-1'H,7H-spiro[furo[3,4-b]pyridine-5,4'-piperidin]-1'-yl)-1,3-dihydro-4'H-spiro[indene-2,5'-[1,3]oxazol]-4'-one

(71) Applicant: Hoffmann-La Roche Inc., Little Falls, NJ (US)

(72) Inventors: Jean-Michel ADAM, Rosenau (FR); Stephan BACHMANN, Allschwil (CH); Raphael BIGLER, Thurgau (CH); Pascal DOTT, Rixheim (FR); Serena Maria FANTASIA, Saint-Louis (FR); Katja GROSSE-SENDER, Allschwil (CH); Philippe JAMES, Saint-Louis (FR); Paul SPURR, Riehen (CH); Juergen THUN, Grenzach-Wyhlen (DE); Sandro TONAZZI, Zollikon (CH); Paolo TOSATTI, Binningen (CH)

(73) Assignee: Hoffmann-La Roche Inc., Little Falls, NJ (US)

(21) Appl. No.: 18/665,776

(22) Filed: May 16, 2024

Related U.S. Application Data
(63) Continuation of application No. PCT/EP2022/082192, filed on Nov. 17, 2022.

(30) Foreign Application Priority Data
Nov. 19, 2021 (EP) 21209197.9

(51) Int. Cl. C07D 491/20

(52) U.S. Cl. CPC C07D 491/20

(57) ABSTRACT

The invention provides crystalline forms of 2′-(7,7-dimethyl-1′H,7H-spiro[furo[3,4-b]pyridine-5,4′-piperidin]-1′-yl)-1,3-dihydro-4′H-spiro[indene-2,5′-[1,3]oxazol]-4′-one, and a process for converting Form A to Form B for use in medicaments for treatment of anxiety, depression, irritability, impaired social interactions and psychomotor coordination, and other indications. The invention further provides processes to manufacture 2′-(7,7-dimethyl-1′H,7H-spiro[furo[3,4-b]pyridine-5,4′-piperidin]-1′-yl)-1,3-dihydro-4′H-spiro[indene-2,5′-[1,3]oxazol]-4′-one. Also disclosed are compounds useful as intermediates in the methods of the invention.

### CROSS REFERENCE TO RELATED APPLICATIONS

This application is a continuation of PCT Application No. PCT/EP2022/082192 filed on Nov. 17, 2022, which claims priority of EP Application No. 21209197.9 filed on Nov. 19, 2021, the disclosures of which are incorporated herein by reference.

### FIELD OF THE INVENTION

The invention provides processes to manufacture 2′-(7,7-dimethyl-1′H,7H-spiro[furo[3,4-b]pyridine-5,4′-piperidin]-1′-yl)-1,3-dihydro-4′H-spiro[indene-2,5′-[1,3]oxazol]-4′-one. Also disclosed are compounds useful as intermediates in the methods of the invention.

### BACKGROUND OF THE INVENTION

The suprachiasmatic nucleus (SCN) is the endogenous clock of the body regulating circadian rhythmicity and is known to be rich in vasopressin neurons (Kalsbeek et al. 2010)¹, producing and releasing vasopressin with a 24 h circadian rhythm (Schwartz et al. 1983)². A major regulatory effect of vasopressin on circadian rhythm could not be demonstrated by the prior art. The Brattleboro rat, a rat strain naturally lacking vasopressin due to a point mutation, has no obvious defect in its circadian rhythm (Groblewski et al. 1981)³. Injection of vasopressin directly in the hamster SCN had no effect on circadian phase shift (Albers et al. 1984)⁴. In contrast, the vasopressin receptors were shown to modulate the circadian clock in a more subtle way. Yamaguchi et al (2013)⁵ demonstrated that V1a knock-out and V1a/V1b double knock-out mice show faster reentrainment to the new light/dark cycle after a circadian phase advance or a phase delay, an experiment mimicking jet-lag in humans. The same result was obtained after chronic administration of a mixture of V1a and V1b small molecule antagonists through a minipump directly on the SCN.

Poor sleep can lead to numerous health disturbances including anxiety, depression, irritability, impaired social interactions and psychomotor coordination and the like.

WO2013/176220 describes circadian rhythm-regulating agents which comprises an inhibitor capable of inhibiting vasopressin receptors V1a and V1b.

2′-(7,7-Dimethyl-1′H,7H-spiro[furo[3,4-b]pyridine-5,4′-piperidin]-1′-yl)-1,3-dihydro-4′H-spiro[indene-2,5′-[1,3]oxazol]-4′-one has previously been described in the art in WO2015/091411.

### SUMMARY OF THE INVENTION

It has been found that by using the processes according to the present invention 2′-(7,7-dimethyl-1′H,7H-spiro[furo[3,4-b]pyridine-5,4′-piperidin]-1′-yl)-1,3-dihydro-4′H-spiro[indene-2,5′-[1,3]oxazol]-4′-one and its pharmaceutically acceptable salts can be prepared more economically with less process steps under moderate reaction conditions with an outstanding yield. Further, crude intermediate products can mostly be used in subsequent reaction steps without the need of any additional purification steps.

Further, two forms have been identified, and it has been further found that Form B is the most preferred one. Up to now the compound of formula (I) has been described only in amorphous form. This form is not suitable for further drug development and the need still exists for a less hygroscopic and thermodynamically stable form at ambient conditions of the compound of formula (I). This problem was surprisingly solved by the crystalline Form B of the compound of formula (I) (hereinafter designated “Form B”).

### BRIEF DESCRIPTION OF THE DRAWINGS

FIG. 1 is an XRPD pattern of Form A.

FIG. 2 is an IR spectrum of form A where % T represents % Transmittance.

FIG. 3 is a Raman spectrum of form A.

FIG. 4 is an XRPD pattern of Form B.

FIG. 5 is an IR spectrum of form B.

FIG. 6 is a Raman spectrum of form B.

### DETAILED DESCRIPTION OF THE INVENTION

### Definitions

The following definitions of the general terms used in the present description apply irrespectively of whether the terms in question appear alone or in combination with other groups.

The term “room temperature” (RT) refers to 18-30° C.

“Solution” as used herein is meant to encompass liquids wherein a reagent or reactant is present in a solvent in dissolved form (as a solute) or is present in particulate, undissolved form, or both. Thus, in a “solution”, it is contemplated that the solute may not be entirely dissolved therein and solid solute may be present in dispersion or slurry form. Accordingly, a “solution” of a particular reagent or reactant is meant to encompass slurries and dispersions, as well as solutions, of such reagents or reactants. “Solution” and “Slurry” may be used interchangeable herein.

“Solvent” as used herein is meant to encompass liquids that fully dissolve a reagent or reactant exposed to the solvent, as well as liquids which only partially dissolve the reagent or reactant or which act as dispersants for the reagent or reactant. Thus, when a particular reaction is carried out in a “solvent”, it is contemplated that some or all of the reagents or reactants present may not be in dissolved form.

The term “approximately” in connection with degrees 2-theta values refers to +0.2 degrees 2-theta.

The terms “crystalline form” or “form” refer to polymorphic forms and solvates of a compound.

The term “pharmaceutically acceptable salts” refers to salts that are suitable for use in contact with the tissues of humans and animals. Examples of suitable salts with inorganic and organic acids are, but are not limited to acetic acid, citric acid, formic acid, fumaric acid, hydrochloric acid, lactic acid, maleic acid, malic acid, methanesulfonic acid, nitric acid, phosphoric acid, p-toluenesulfonic acid, succinic acid, sulfuric acid, tartaric acid, trifluoroacetic acid and the like. Preferred are formic acid, trifluoroacetic acid and hydrochloric acid. Most preferred is hydrochloric acid.

The term “phase shift sleep disorders” summarizes conditions classified as disturbances in the circadian rhythm, i.e. the approximately 24-hour cycles that are generated by an organism, e.g. a human being.

Phase shift sleep disorders include, but are not limited to transient disorders like jetlag or a changed sleep schedule due to work, social responsibilities, or illness, as well as chronic disorders like delayed sleep-phase syndrome (DSPS), delayed sleep-phase type (DSPT), advanced sleep-phase syndrome (ASPS), and irregular sleep-wake cycle.

The nomenclature used in this Application is based on IUPAC systematic nomenclature, unless indicated otherwise.

The invention is provides a process to synthesize a crystalline form of a compound of formula I

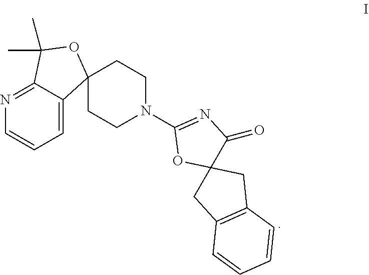 [SMILES: CC1(C)OC2(CCN(C3=NC(=O)C4(Cc5ccccc5C4)O3)CC2)c2cccnc21]

A certain embodiment of the invention relates to the crystalline Form A of the compound of formula I as described herein, characterized by a X-ray powder diffraction pattern having the characteristic peaks expressed in values of degrees 2-theta at approximately (+0.20 degree 2-theta)

degree 2-theta

9.25
9.77
11.13
12.85
13.32
14.48
14.86
17.00
17.37
17.87
18.55
19.60
21.15
21.43
21.63
22.35
22.95
23.37
24.26
24.48

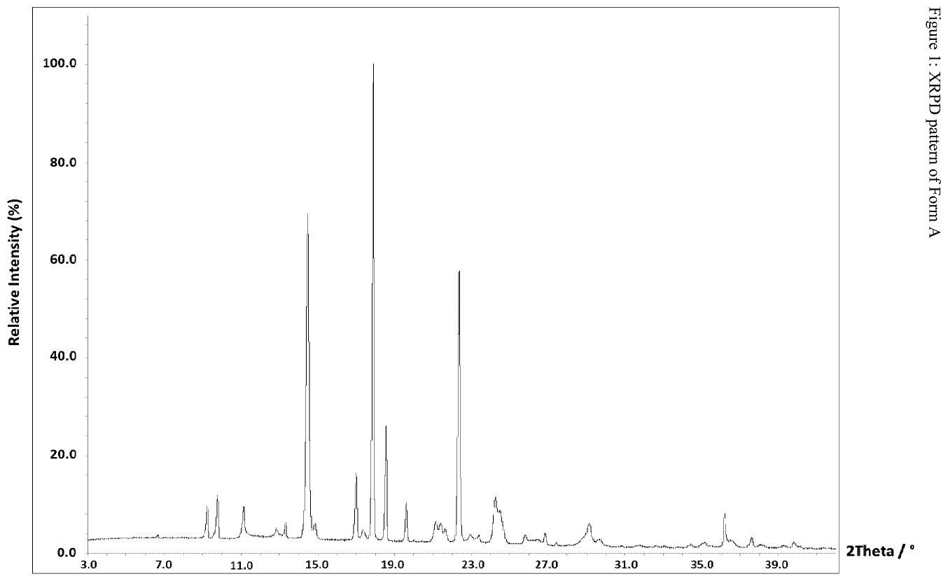

A certain embodiment of the invention relates to the crystalline Form A of the compound of formula I as described herein, characterized by the X-ray powder diffraction pattern as shown in FIG. 1.

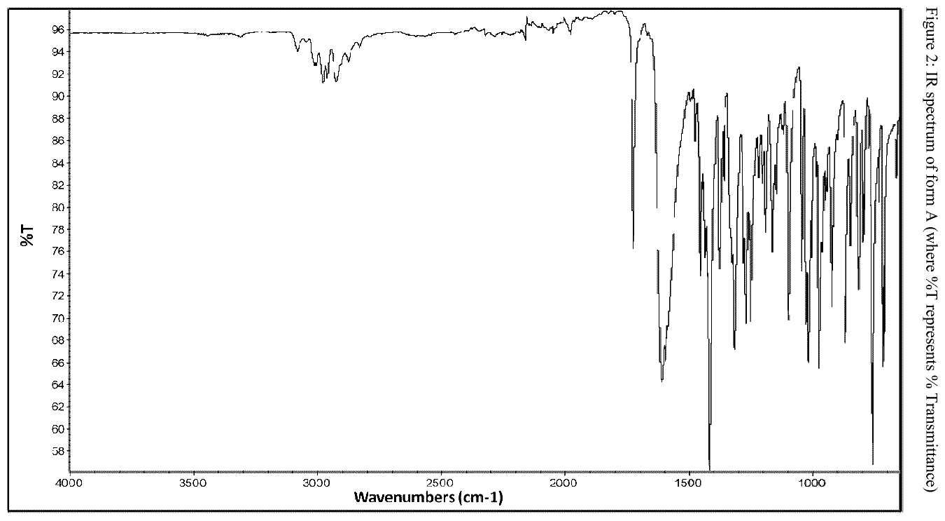

A certain embodiment of the invention relates to the crystalline Form A of the compound of formula I as described herein, characterized by the Infrared spectrum shown in as shown in FIG. 2.

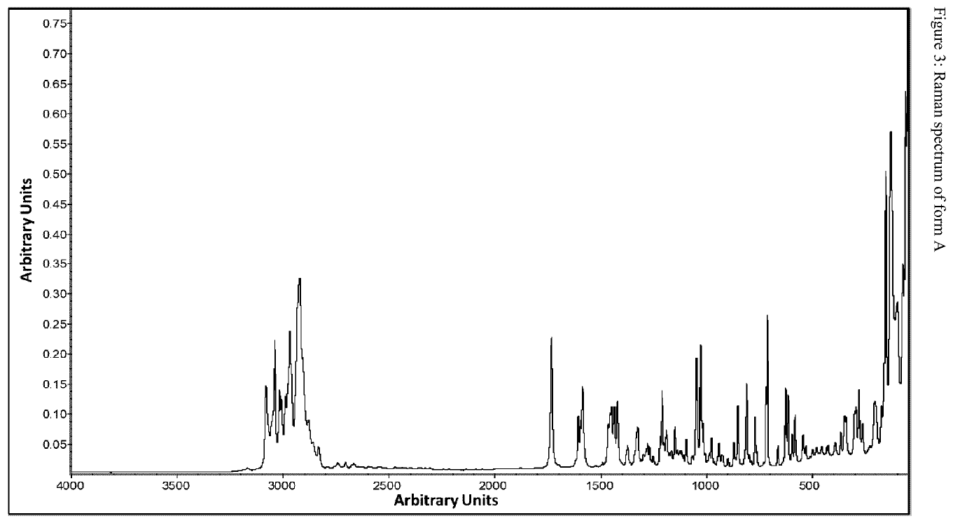

A certain embodiment of the invention relates to the crystalline Form A of the compound of formula I as described herein, characterized by the Raman spectrum shown in as shown in FIG. 3.

A certain embodiment of the invention relates to the crystalline Form B of the compound of formula I as described herein, characterized by a X-ray powder diffraction pattern having the characteristic peaks expressed in values of degrees 2-theta at approximately (+0.20 degree 2-theta)

degree 2-theta

6.66
9.26
9.77
11.12
12.84
13.11
13.34
14.33
14.64
17.01
17.20
17.60
17.88
18.20
18.58
19.61
20.07
21.46
22.33
22.76
23.24
24.06
24.42
24.77
25.84
26.87
28.88
30.91
32.36
34.49
36.21
37.64
39.82

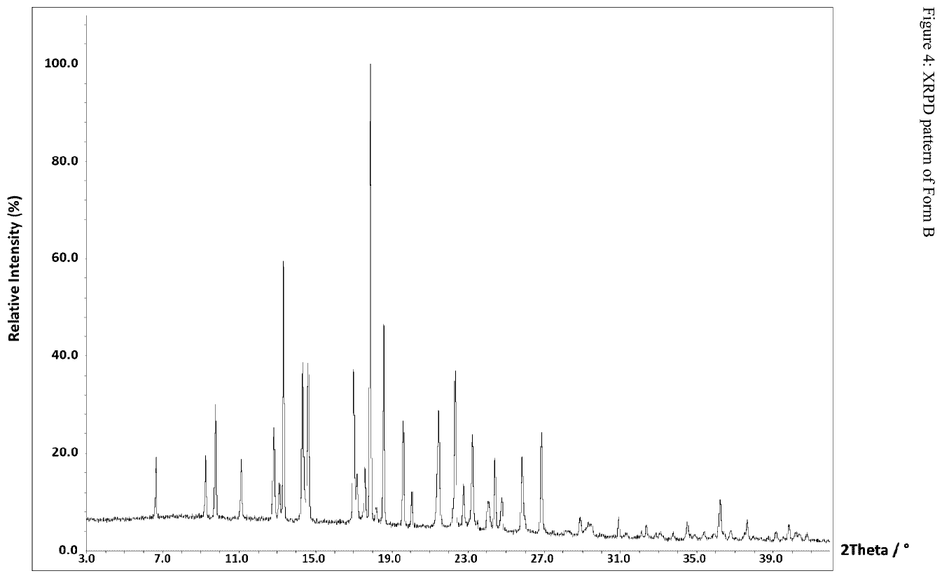

A certain embodiment of the invention relates to the crystalline Form B of the compound of formula I as described herein, characterized by the X-ray powder diffraction pattern as shown in FIG. 4.

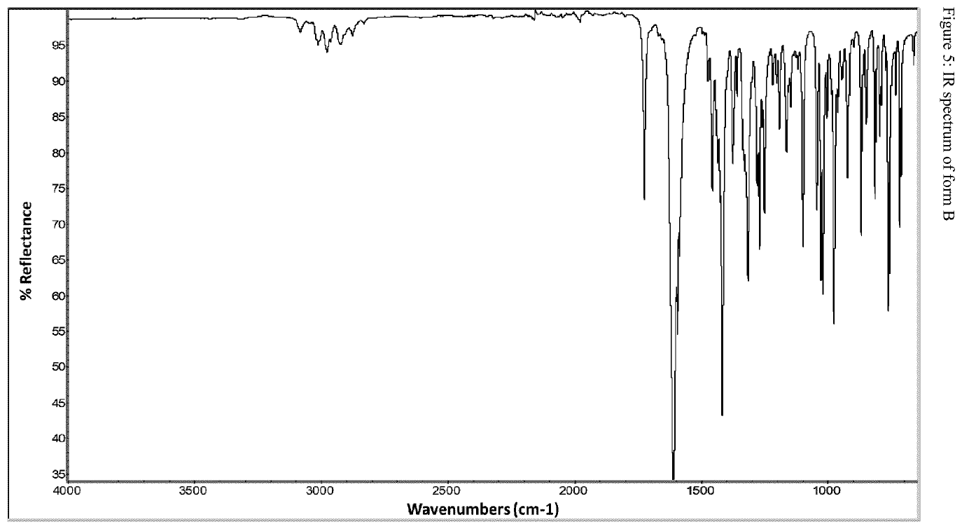

A certain embodiment of the invention relates to the crystalline Form B of the compound of formula I as described herein, characterized by the Infrared spectrum shown in as shown in FIG. 5.

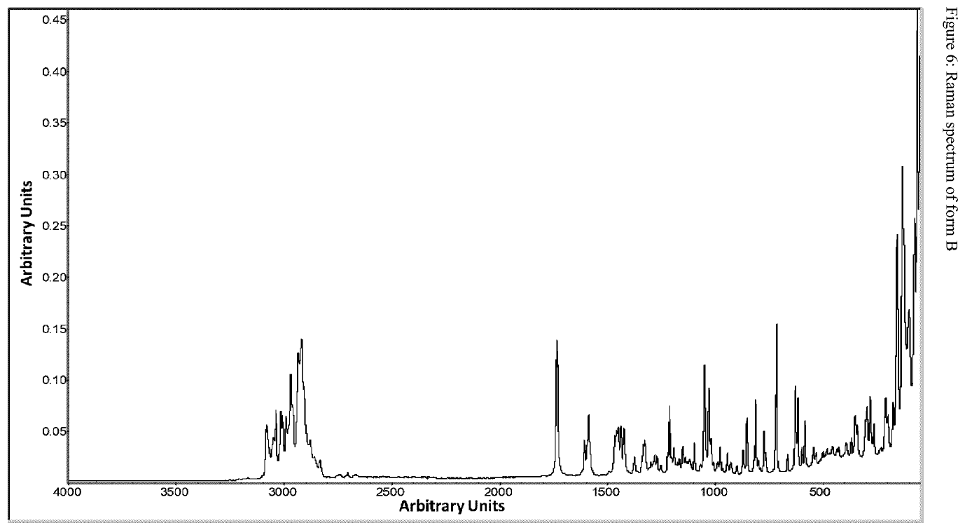

A certain embodiment of the invention relates to the crystalline Form B of the compound of formula I as described herein, characterized by the Raman spectrum shown in as shown in FIG. 6.

The invention further relates to a process to transform Form A to Form B.

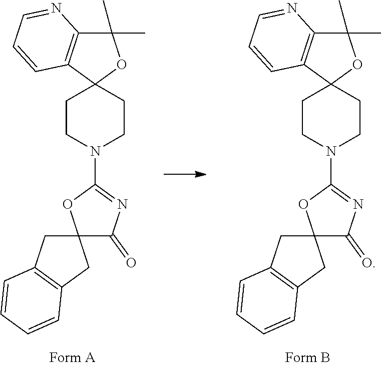 [SMILES: CC1(C)OC2(CCN(C3=NC(=O)C4(Cc5ccccc5C4)O3)CC2)c2cccnc21 ; CC1(C)OC2(CCN(C3=NC(=O)C4(Cc5ccccc5C4)O3)CC2)c2cccnc21]

Form B is the thermodynamically stable known crystalline form of the compound of formula (I) at ambient temperature.

A certain embodiment of the invention relates to a process to synthesize a compound of formula I as described herein, comprising reacting a compound of formula TT with a compound of formula IX

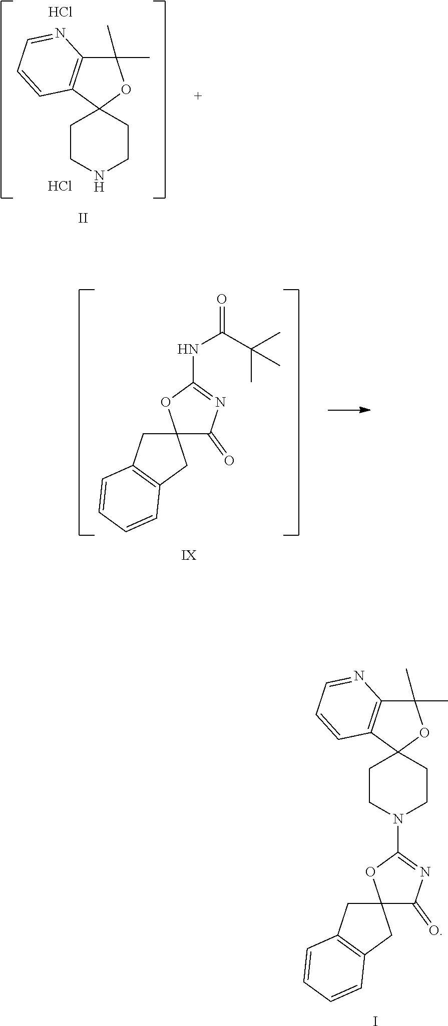

A certain embodiment of the invention relates to the transformation above, wherein the 7,7-dimethylspiro[furo[3,4-b]pyridine-5,4′-piperidine] dihydrochloride is added to the 2,2-dimethyl-N-(4′-oxospiro[indane-2,5′-oxazole]-2′-yl)propanamide suspension at a temperature of 50-60° C. and allowed to react at 55° C. in the presence of N,N-diisopropylethylamine.

A certain embodiment of the invention relates to a process to synthesize a compound of formula I comprising the following steps:

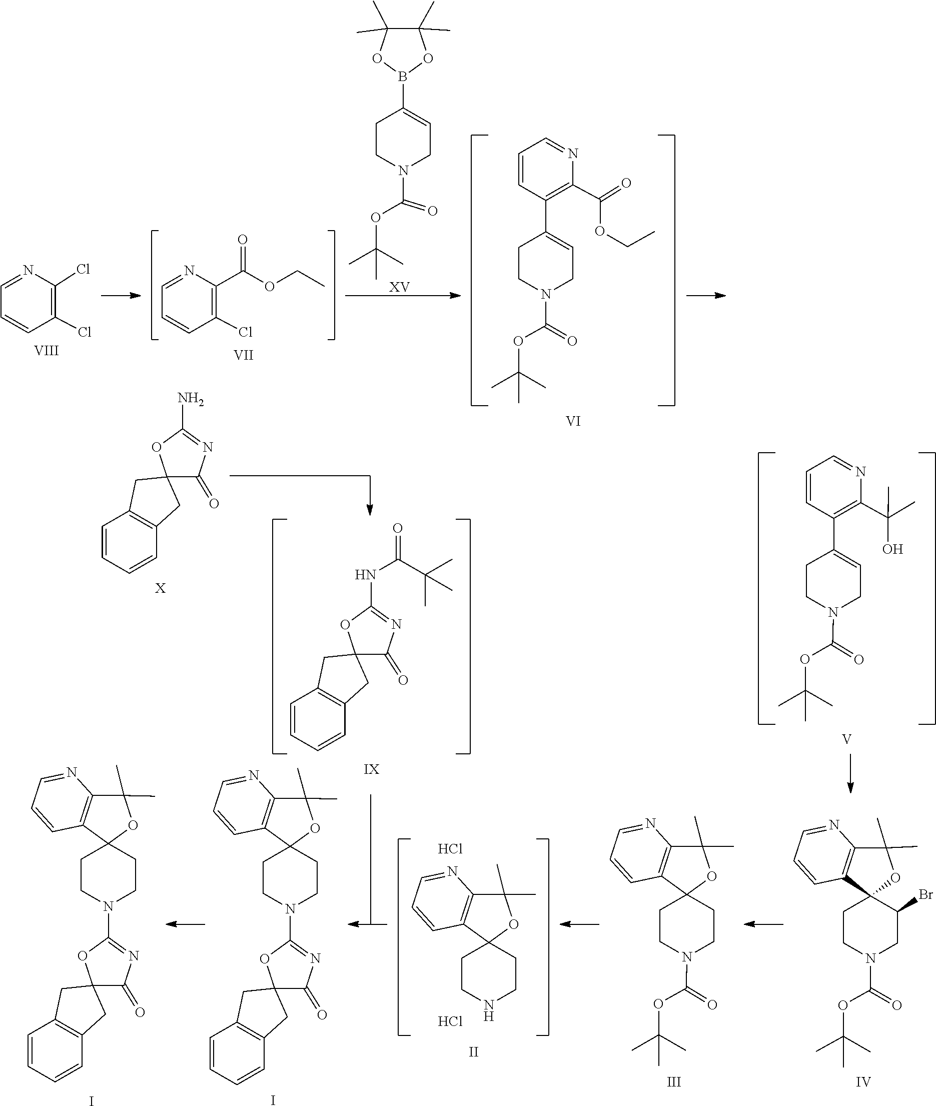

A certain embodiment of the invention relates to a process comprising reacting a compound of formula X to a compound of formula IX:

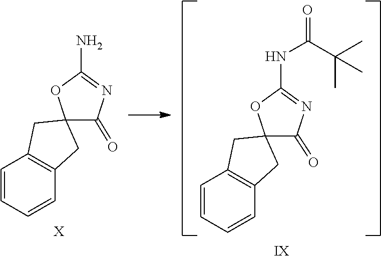

A certain embodiment of the invention relates to the process above, which takes place in the presence of acetonitrile, pyridine and pivaloylchloride at a temperature of 60° C.

A certain embodiment of the invention relates to the process to synthesize a compound of formula X, comprising the following steps:

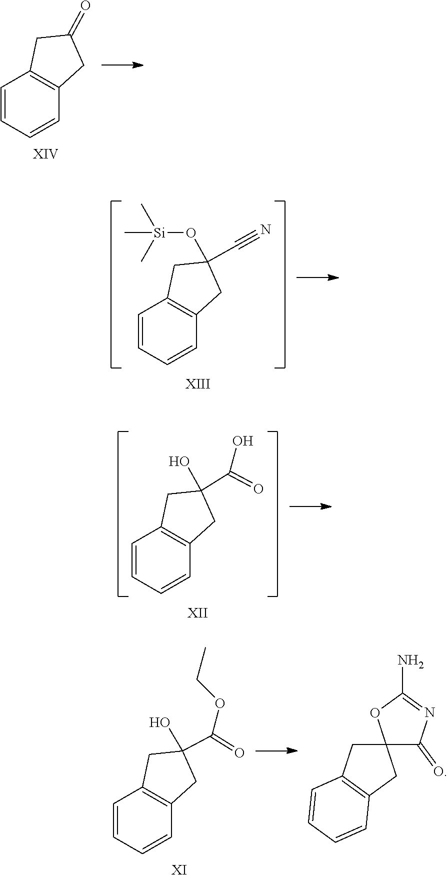

A certain embodiment of the invention relates to the compound of formula (XI), which is particularly advantageous in that it gives an easy and convenient access to the compound of formula (X)

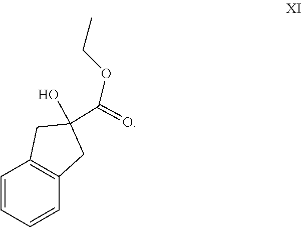

The known synthesis of the compound of formula (I) involves many steps, sometimes with limited yields. This problem has been addressed and improvements achieved through the provision of the compound and process of the invention, and thereafter a benefit resulting in less waste and increased yield and process robustness was achieved. According to the process of the invention, the compound of formula XI is thus obtained from Scheme below.

A certain embodiment of the invention relates the process comprising reacting a compound of formula XIV to a compound of formula XI via the steps found in the following steps:

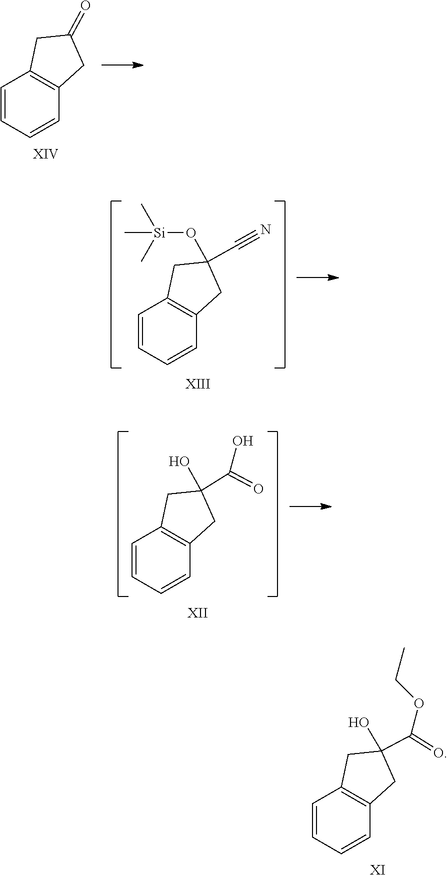

A certain embodiment of the invention relates to the process as described above, wherein the compound of formula XIV is reacted in a solution of trimethylsilylcyanide in methylene chloride in the presence of zinc iodide at 10 to 20° C.

A certain embodiment of the invention relates to the process described above, wherein the compound of formula XII is obtained by hydrolysis of compound of formula XIII using concentrated hydrochloric acid in toluene at 80 to 90° C.

A certain embodiment of the invention relates to the process described above, wherein the compound of formula XI is obtained through esterification of compound of formula XII.

A certain embodiment of the invention relates to the process comprising reacting a compound of formula VIII to a compound of formula VII.

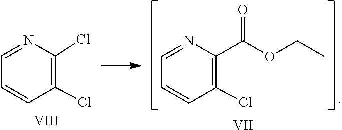

A certain embodiment of the invention relates to the process as described above, wherein the compound of formula VIII is treated in the presence of palladium catalyst, ethanol and an organic base under pressure of carbon monoxide atmosphere at 100° C. Using an organic base versus the typical inorganic base of Sodium Acetate⁶ increased the throughput of the process by a factor of approx. 5 and provided for a reduced catalytic loading by a factor of up to 10 times.

A certain embodiment of the invention relates to the process as described above, wherein the compound of formula VIII is treated in the presence of palladium catalyst, ethanol and triethylamine under pressure of carbon monoxide atmosphere at 100±5° C.

A certain embodiment of the invention relates to the process as described above, wherein the compound of formula VIII is treated in the presence of PdCl₂(dppp) (dppp known as 1,3-Bis(diphenylphosphino)propane), ethanol and an organic base under pressure of carbon monoxide atmosphere at 100±5° C.

A certain embodiment of the invention relates to the process as described above, wherein the compound of formula VIII is treated in the presence of PdCl₂(dppp), ethanol and triethylamine under pressure of carbon monoxide atmosphere at 100±5° C.

A certain embodiment of the invention relates to the process as described above, wherein the compound of formula VIII is treated in the presence of PdCl₂(dppp), ethanol and triethylamine under 60-100 bar pressure of carbon monoxide atmosphere at 100±5° C. While literature provides for 15-25 bar of carbon monoxide atmosphere^{6,7}, it was found that higher regioselectivity was achieved with lower levels of diester formation through increasing the pressure (at 20 bar approximately 3% of diester were formed).

A certain embodiment of the invention relates to the process as described above, wherein the compound of formula VIII is treated in the presence of PdCl₂(dppp), ethanol and triethylamine under 60-100 bar pressure of carbon monoxide atmosphere at 100±5° C. and a substrate/catalyst ratio (S/C) greater than 100 and up to 1000.

A preferred embodiment of the invention relates to the process as described above, wherein the compound of formula VIII is treated in the presence of PdCl₂(dppp), ethanol and triethylamine under 60-80 bar pressure of carbon monoxide atmosphere at 100±5° C. and a substrate/catalyst ratio (S/C) greater than 100 and up to 1000.

A certain embodiment of the invention relates to the process comprising reacting a compound of formula VII with a compound of XV to a compound of formula VI.

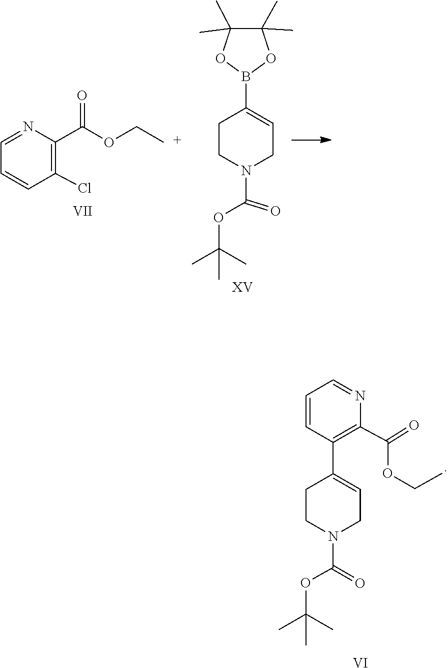

A certain embodiment of the invention relates to the process described above, wherein the reaction takes place in the presence of a palladium catalyst via a Suzuki-Miyaura coupling reaction.

A certain embodiment of the invention relates to the process described above, wherein the palladium catalyst comprises a palladium precursor and a ligand.

A certain embodiment of the invention relates to the process described above, wherein the ligand is a monophosphine of general structure L=P(R)(R¹)₂ where R=tert-butyl, n-butyl, 1-phenyl-1H-pyrrol-2-yl or 4-dimethylaminophenyl and R¹=cyclohexyl, tert-butyl or adamantyl. More specifically R=4-dimethylaminophenyl and R¹=tert-butyl (L=4-(di-tert-butylphosphino)-N,N-dimethylaniline).

A certain embodiment of the invention relates to the process described above, wherein the palladium precursor is a palladium (II) or a palladium (0) specie such as: [PdCl(X)]₂ (X=e.g., allyl, cinnamyl, crotyl, indenyl), [Pd(X)(Y)] (Y=e.g., cyclopentadienyl or p-cymyl), Pd(dba)₂, Pd₂(dba)₃, Pd(OAc)₂, PdZ₂ (Z=Cl, Br, I), and Pd(TFA)₂. Pd(MeCN)₂Cl₂, Pd(benzonitrile)₂Cl₂, Pd(MeCN)₄(BF₄)₂, Pd(acac)₂, di-g-chlorobis[2′-(amino-N)[1,1′-biphenyl]-2-yl-C]dipalladium(II), di-μ-mesylbis[2′-(amino-N)[1,1′-biphenyl]-2-yl-C]dipalladium(II), di-μ-chlorobis[2-[(dimethylamino)methyl]phenyl-C,N]dipalladium(II). More specifically the palladium precursor is Pd(OAc)₂.

The palladium precursor could also be a palladium complex containing an AmPhos ligand such as: chloro[4-(di-tert-butylphosphino)-N,N-dimethylaniline-2-(2′-aminobiphenyl)]palladium(II), [4-(Di-tert-butylphosphino)-N,N-dimethylaniline-2-(2′-aminobiphenyl)]palladium(II) methanesulfonate, [4-(Di-tert-butylphosphino)-N,N-dimethylaniline-2-(2′-N-methylaminobiphenyl)]palladium(II) methanesulfonate, [Pd(AmPhos)₂Cl₂], [Pd(AmPhos)₂], [Pd(AmPhos)Cl(X)] ((X=e.g., allyl, cinnamyl, or crotyl, indenyl), [Pd(AmPhos)(X)]OTf. In such case an additional ligand is not necessary required.

A certain embodiment of the invention relates to the process described above, wherein the reaction takes place in the presence of a palladium acetate with triphenylphosphine or 4-(di-tert-butylphosphanyl)-N,N-dimethylaniline.

A certain embodiment of the invention relates to the process described above, wherein the reaction takes place in the presence of palladium acetate with 4-(di-tert-butylphosphanyl)-N,N-dimethylaniline. Such a combination of catalyst and ligand was shown to be a more active catalyst system, requiring 7.5 times less Pd-Catalyst and 12 times less phosphine then the Pd(OAc)₂/triphenylphosphine system, creating an economic advantage and requiring less effort to deplete the residual palladium in the downstream process.

A certain embodiment of the invention relates to the process described above, wherein the reaction takes place in the presence of a palladium acetate with 4-(di-tert-butylphosphanyl)-N,N-dimethylaniline and tetrabutylammonium bromide.

A certain embodiment of the invention relates to the process described above, wherein the reaction takes place in the presence of a palladium acetate with 4-(di-tert-butylphosphanyl)-N,N-dimethylaniline and tetrabutylammonium bromide at 110° C. in tert-amyl alcohol as solvent.

A certain embodiment of the invention relates to the process comprising reacting a compound of VI to a compound of V:

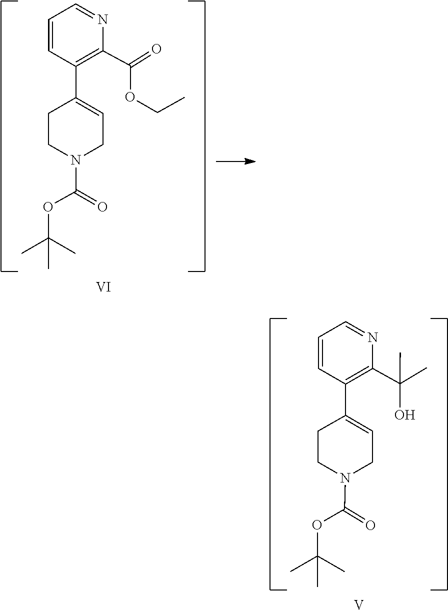

A certain embodiment of the invention relates to the process described above, wherein VI was added onto the Grignard reagent methylmagnesium bromide to generate V.

A certain embodiment of the invention relates to the process comprising reacting a compound of VI to a compound of V-MgCl:

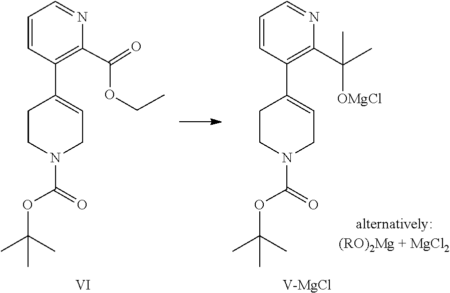

A certain embodiment of the invention relates to the process described above, wherein VI was reacted with methylmagnesium chloride in THF.

A certain embodiment of the invention relates to the process comprising reacting a compound of V-MgCl to a compound of IV.

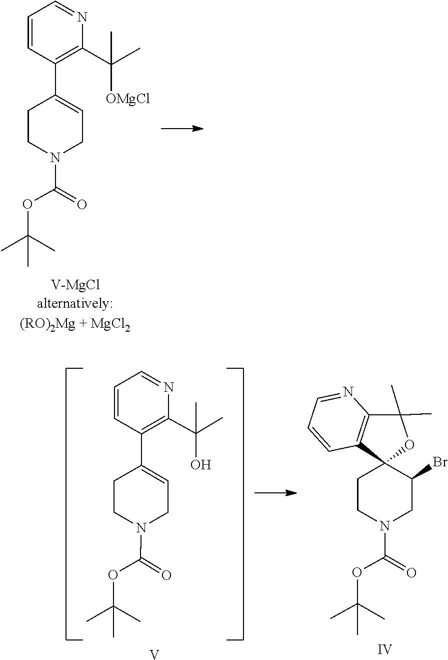

A certain embodiment of the invention relates to the process comprising reacting a compound of IV to a compound of III.

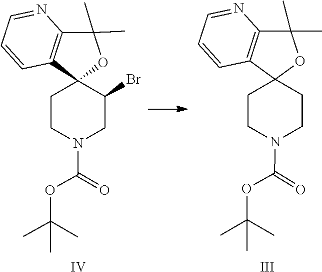

A certain embodiment of the invention relates to a compound of formula I or a pharmaceutically acceptable salt, whenever prepared by a process as described herein.

A certain embodiment of the invention relates to the intermediate IX

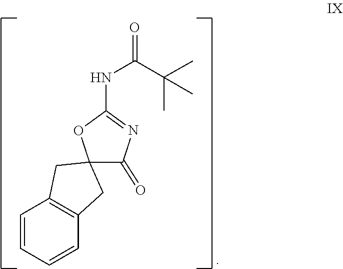

A certain embodiment of the invention relates to the process to synthesize a compound of formula I, whereby a compound of formula IX is formed as an intermediate.

A certain embodiment of the invention relates to the process to synthesize a compound of formula I, whereby a compound of formula II is formed as an intermediate.

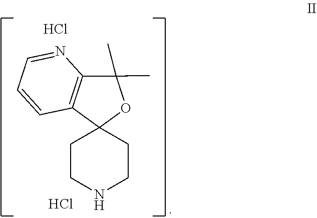

A certain embodiment of the invention relates to a compound of formula I as described herein for use as a medicament.

A certain embodiment of the invention relates to a compound of formula I as described herein for use in the therapeutic and/or preventive treatment of inappropriate secretion of vasopressin, anxiety, depressive disorders, obsessive compulsive disorder, autistic spectrum disorders, schizophrenia, aggressive behavior, phase shift sleep disorders, in particular jetlag, or circadian disorders.

### Experimental Part

The following experiments are provided for illustration of the invention. They should not be considered as limiting the scope of the invention, but merely as being representative thereof.

### IR Analysis

The ATR FTIR spectra were recorded without any sample preparation using a ThermoNicolet iS5 FTIR spectrometer with ATR accessory. The spectral range is between 4000 cm⁻¹ and 650 cm⁻¹ resolution 2 cm⁻¹ and 50 co-added scans were collected. Happ-Genzel apodization was applied. Using ATR FTIR will cause the relative intensities of infrared bands to differ from those seen in a transmission FTIR spectrum using KBr disc or nujol mull sample preparations. Due to the nature of ATR FTIR, the bands at lower wavenumber are more intense than those at higher wavenumber.

### Raman Analysis

The FT-Raman spectrum was collected in the spectral range of 4000-50 cm⁻¹ with a Bruker MultiRam FT-Raman spectrometer, equipped with a NdYAG 1064 nm laser and a liquid nitrogen cooled Germanium detector. The laser power was set to 300 mW, 2 cm⁻¹ resolution was used and 1024 scans were co-added. The apodization used was Blackman-Harris 4-term.

### X-Ray Analysis

X-ray diffraction patterns were recorded at ambient conditions in transmission geometry with a STOE STADI P diffractometer (Cu Kai radiation, primary monochromator, silicon strip detector, angular range 3° to 42° 2Theta, 0.02° 2Theta step width and 20 seconds measurement time per step. The samples are prepared and analyzed without further processing (e.g. grinding or sieving) of the substance.

### Form A of I

Form A can be obtained by fast evaporative crystallization of I from dichloromethane or by fast evaporative crystallization of I from chloroform.

### Form B of I

197 mg of Form A of I was suspended in a closed vial at ambient temperature in 2 mL of 1-propanol. After 14 days stirring time at 22° C. the solid was isolated by centrifugation (10 min/22° C./1500 rpm). The sample was dried for approx. 48 h at 50° C./<5 mbar.

### 2-Hydroxyindane-2-carboxylic acid

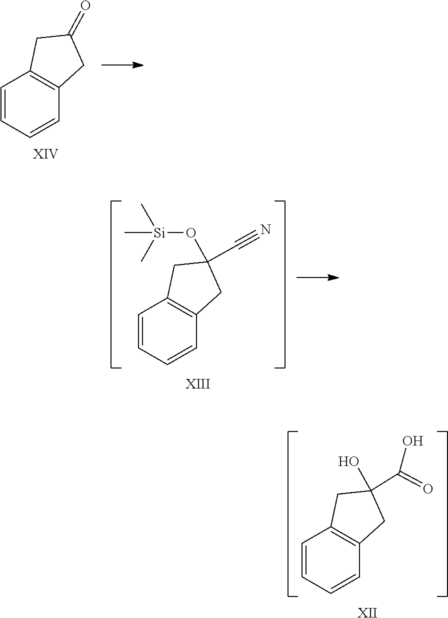

Under nitrogen atmosphere, indan-2-one (33.0 kg, 250 mol, Eq: 1.00), methylene chloride (132 kg, 99 L) and zinc iodide (0.792 kg, 2.48 mol, Eq: 0.010) were charged at room temperature in a reactor. A solution of trimethylsilyl cyanide (28.05 kg, 283 mol, Eq: 1.13) in methylene chloride (33.0 kg, 24.8 L) was added within 3 h to the mixture at 10 to 20° C. and the reaction was stirred at this temperature for 4 h. A 2% aqueous sodium hypochlorite solution (198 kg) was added at 10 to 20° C. and the mixture stirred at this temperature for 2 h. The phases were separated, the organic layer washed with water (168 kg) and then concentrated under reduced pressure at a jacket temperature of 35 to 45° C. Toluene (132 kg) and then concentrated hydrochloric acid (66 kg) were added. The mixture was heated and stirred at 80 to 90° C. for 5 h. The mixture was cooled to 0 to 10° C. and stirred for 1 h. The suspension was filtered and the cake was washed with toluene (16.5 kg) affording 32.8 kg of the title compound as a wet cake.

### Ethyl 2-hydroxyindane-2-carboxylate

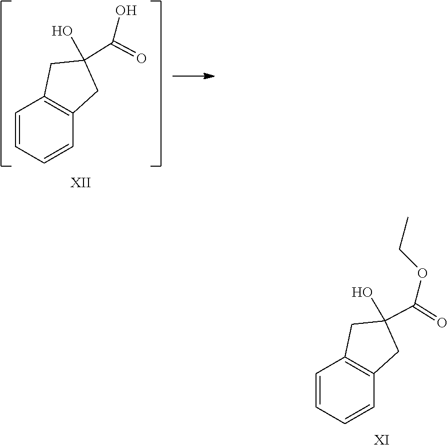

Under nitrogen atmosphere, wet 2-hydroxyindane-2-carboxylic acid (32.8 kg) and toluene (154 kg, 177 L) were charged in a reactor and water was removed by azeotropic distillation under strong refluxing conditions. After cooling to 30 to 40° C., sulfuric acid (0.55 kg, 5.5 mol, Eq: 0.02) and ethanol (39.1 kg, 49.5 L) were charged and the mixture stirred at 110 to 120° C. for 12 h. The mixture was cooled to 30 to 40° C. and filtered. The obtained solution was concentrated under reduced pressure to 1.0 to 1.5 volumes. n-heptane (61.5 kg, 90.4 L) was charged and a 1% aqueous solution of sodium bicarbonate (154 kg) added at 10 to 20° C. over 2 to 3 h, then stirred for 3 h at 0 to 10° C. The suspension was filtered and the cake washed with n-heptane (15.4 kg, 22.6 L) and dried under vacuum at 40° C. providing 26.15 kg of the title compound as a yellow solid.

### 2′-Aminospiro[indane-2,5′-oxazole]-4′-one

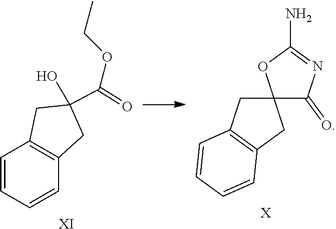

Under nitrogen atmosphere, ethyl 2-hydroxyindane-2-carboxylate (26.15 kg, 127 mol, Eq: 1.00), ethanol (91.5 kg, 116 L) and guanidine hydrochloride (13.9 kg, 146 mol, Eq: 1.15) were charged in a reactor. 30% Sodium methoxide in methanol (25.4 kg, 141 mol, Eq: 1.11) was added at 10 to 20° C. over 2 h and stirred at this temperature for 10 h. The mixture was concentrated under reduced pressure to 1.0 to 1.5 volumes. 1.3% aqueous hydrochloric acid solution (131 kg) was added at 0 to 10° C. to achieve pH=2.0 to 2.5. The suspension was stirred at 10 to 20° C. for 3 h, filtered and the cake washed with water (26.2 kg). The wet cake was digested with water (131 kg) at 10 to 20° C. for 3 h, filtered and the wet cake washed with water (26.2 kg). The wet cake was digested with tetrahydrofuran (52.3 kg, 58.8 L) at 30 to 40° C. for 2 h, filtered and the wet cake was washed with tetrahydrofuran (13.1 kg, 14.7 L). The wet cake was dried under vacuum at 50° C. furnishing 15.19 kg of the title compound as an off-white solid.

### Ethyl 3-chloropyridine-2-carboxylate

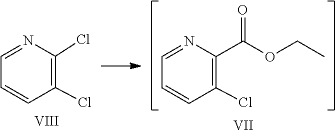

Under an argon atmosphere, 2,3-dichlorpyridine (150 g, 1.01 mol, Eq: 1.00), ethanol (592 g, 750 mL), PdCl₂(dppp) (1.20 g, 2.03 mmol, Eq: 0.0020) and triethylamine (205 g, 2.03 mol, Eq: 2.00) were charged into an autoclave. The atmosphere was exchanged three times with carbon monoxide and the pressure adjusted to 10 bar. Stirring was started and the mixture heated to 100° C. The carbon monoxide pressure was adjusted to 60 bar and the reaction mixture stirred at 100° C. for 20 h. The contents were cooled to room temperature and the atmosphere exchanged five times with argon. The autoclave was discharged and rinsed with ethanol (158 g, 200 mL). The crude mixture was added to a solution of citric acid (97.3 g, 0.506 mol; Eq: 0.50) in water (367 g, 367 mL) with the aid of an addition funnel and the addition funnel was rinsed with ethanol (39.5 g, 50 mL). The solution was filtered and water (985 g, 985 mL) added. Ethanol was removed under reduced pressure and ethyl acetate (630 g, 700 mL) added. Volatiles were removed under reduced pressure and ethyl acetate (1.42 kg, 1.58 L) and water (329 g, 329 mL) were added. The aqueous phase was separated and the organic phase fully concentrated under vacuum. The residue was dissolved in 2-methyl-2-butanol (639 g, 790 mL) and fully concentrated and dried at 55° C./<10 mbar, providing 173.3 g of the title compound as a red-brown oil.

### Ethyl 3-(1-tert-butoxycarbonyl-3,6-dihydro-2H-pyridin-4-yl)pyridine-2-carboxylate

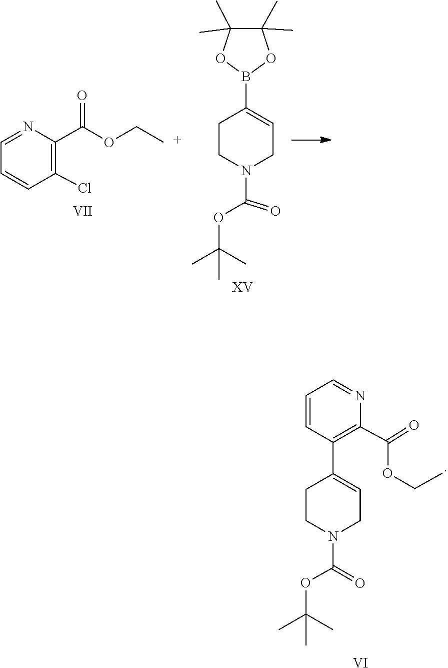

Under an argon atmosphere, tert-butyl 4-(4,4,5,5-tetramethyl-1,3,2-dioxaborolan-2-yl)-3,6-dihydro-2H-pyridine-1-carboxylate (64.4 g, 208 mmol, Eq: 1.00), potassium carbonate (57.6 g, 416 mmol, Eq: 2.00), tetrabutylammonium bromide (3.36 g, 10.4 mmol, Eq: 0.050) and degassed 2-methyl-2-butanol (100 g, 124 mL) were charged in a reactor. Ethyl 3-chloropyridine-2-carboxylate as a 25.2 w/w % solution in degassed 2-methyl-2-butanol (153.2 g, 208 mmol, Eq: 1.00) was added with the aid of 2-methyl-2-butanol (93.5 g, 116 mL). In a separate flask under an argon atmosphere, 4-(di-tert-butylphosphanyl)-N,N-dimethylaniline (829 mg, 3.12 mmol, Eq: 0.015) and palladium acetate (467 mg, 2.08 mmol, Eq: 0.010) were suspended in degassed 2-methyl-2-butanol (24.2 g, 30 mL) and stirred for 15 minutes. The catalyst solution was added to the reactor and the mixture heated to reflux at 102° C. for 2 h. After cooling to room temperature, tert-butyl methyl ether (430 g, 582 mL) and water (716 g, 716 mL) were added. The aqueous layer was separated and a solution of sodium chloride (31.2 g) in water (624 g, 624 mL) was added. The aqueous layer was separated and the organic layer filtered sequentially through a Speedex pad and an active charcoal filter. The reactor and the filters were rinsed with tert-butyl methyl ether (68 g, 92 mL). The organic solution was stripped of solvent under reduced pressure, toluene (116 g, 134 mL) was added and the solution evaporated again to dryness under reduced pressure. 2-Methyltetrahydrofuran (110 g, 129 mL) was added to the orange residue (190.2 g) creating a 32.6 w/w % solution of title compound in 2-methyltetrahydrofuran.

### tert-Butyl 4-[2-(1-hydroxy-1-methyl-ethyl)-3-pyridyl]-3,6-dihydro-2H-pyridine-1-carboxylate

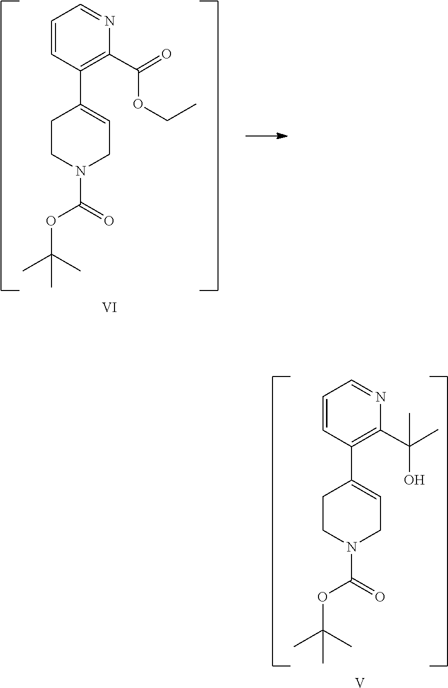

Under a nitrogen atmosphere, 2-methyltetrahydrofuran (192 g, 230 mL) was charged in a reactor and 21.9 w/w % methylmagnesium chloride in tetrahydrofuran (263.1 g, 261 mL, 770 mmol, Eq: 3.70) was added at room temperature via an addition funnel, which was rinsed with 2-methyltetrahydrofuran (23.9 g, 28 mL). tert-Butyl 4-[2-(1-hydroxy-1-methyl-ethyl)-3-pyridyl]-3,6-dihydro-2H-pyridine-1-carboxylate as a 36.3 w/w % solution in 2-methyl-tetrahydrofuran (190.2 g, 69.0 g corrected, 208 mmol, Eq: 1.00) was added at 15-25° C. using an addition funnel and the addition funnel was rinsed with 2-methyltetrahydrofuran (25 g, 29.3 mL). The beige suspension was cooled to 10° C. and a solution of acetic acid (92.5 g, 1.54 mol, Eq: 7.41) in tert-butyl methyl ether (285 g, 385 mL) added over three hours at 10-25° C. Water Ca (300 g, 300 mL) followed by tert-butyl methyl ether (300 g, 405 mL) were added at 10-25° C. The aqueous layer was separated and the organic layer filtered sequentially through a Speedex filter and an active charcoal filter at room temperature. The reactor and the filters were rinsed with tert-butyl methyl ether (37 g, 50 mL). The organic layer was extracted once with a solution of citric acid (106 g, 554 mmol, Eq: 2.65) in water (366 g, 366 mL) and three times with a solution of citric acid (53 g, 277 mol, Eq: 1.33) in water (183 g, 183 mL). The aqueous layers were combined and charged into a reactor and the transfer equipment was rinsed with water (50 g, 50 mL). To the aqueous layer, tert-butyl methyl ether (300 g, 405 mL) was added and a 28 w/w % sodium hydroxide aqueous solution (573 g, 441 mL, 4.01 mol, Eq: 19.3) added at 10-25° C. The aqueous layer was separated and the organic layer was washed with water (100 g, 100 mL). The organic solution was concentrated fully under reduced pressure. Acetonitrile (151 g, 192 mL) was added to the residue and the solution was completely evaporated once again under reduced pressure. Acetonitrile (151 g, 192 mL) was added to the residue (350 g) and the solution obtained was passed through a 1 μm filter furnishing the title compound as a 16.7 w/w % solution in acetonitrile.

### tert-Butyl rac-(3'S,5R)-3′-bromo-7,7-dimethyl-spiro[furo[3,4-b]pyridine-5,4′-piperidine]-1′-carboxylate

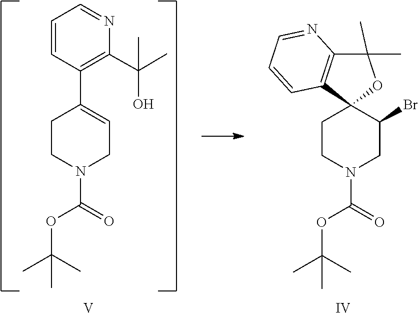

Under an argon atmosphere, N-bromosuccinimide (30.0 g, 167 mmol, Eq: 1.00) was dissolved in acetonitrile (190 g, 242 mL) and using an addition funnel, the solution was added over 75 min at room temperature to a 16.7 w/w % solution of tert-butyl 4-[2-(1-hydroxy-1-methyl-ethyl)-3-pyridyl]-3,6-dihydro-2H-pyridine-1-carboxylate in acetonitrile (318 g, 53.2 g corrected, 167 mmol, Eq: 1.00). The addition funnel was rinsed with acetonitrile (25 g, 32 mL). The mixture was stirred for 15 minutes at room temperature. Ascorbic acid (588 mg, 3.34 mmol, Eq: 0.020) was dissolved in water (57 g, 57 mL) and the solution added via an addition funnel to the reaction mixture at room temperature. The addition funnel was rinsed with water (25 g, 25 mL). Sodium bicarbonate (842 mg, 10.0 mmol, Eq: 0.060) was dissolved in water (81 g, 81 mL) and the solution added with the aid of an addition funnel to the reaction mixture at room temperature. The addition funnel was rinsed with water (25 g, 25 mL). The mixture was concentrated under reduced pressure to a volume of ca. 300 mL and tert-butyl methyl ether (259 g, 350 mL) was added. The aqueous layer was separated and the organic layer concentrated to a volume of ca. 180 mL. Ethanol (473 g, 600 mL) was added and the mixture concentrated under reduced pressure to a volume of ca. 380 mL. Water (290 g, 290 mL) was added over one hour to the solution, maintained at 40° C. The resulting light turbid solution was seeded and stirred for one hour at 40° C. The suspension was cooled to 20° C. over two hours and stirred overnight. Water (250 g, 250 mL) was added over one hour, and the suspension was aged at 20° C. for four hours then filtered. The filter cake was washed with a mixture of ethanol (24 g, 30 mL) and water (90 g, 90 mL) and dried overnight at 50° C./<10 mbar. The title compound (57.9 g) was obtained as white crystals.

### Magnesium 2-(1′-(tert-butoxycarbonyl)-1′,2′,3′,6′-tetrahydro-[3,4′-bipyridin]-2-yl)propan-2-olate chloride

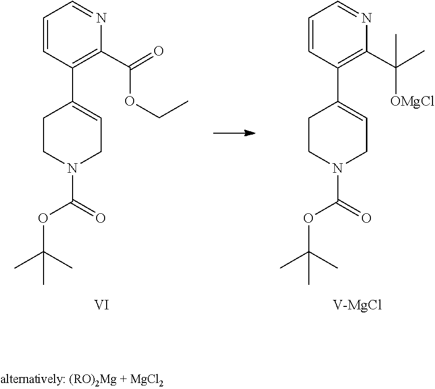

Tetrahydrofuran (8.8 kg, 10 L) was charged in the reactor and cooled to 5° C. Methylmagnesium chloride 22% in THF (6.8 kg, 20 mol, Eq: 2.67) was added over 20 min keeping the temperature between 5-20° C. The feed line was rinsed with tetrahydrofuran (2.64 kg, 3 L). The solution was heated to 20° C. A 31.5% solution of 1′-(tert-butyl)-2-ethyl-3′,6′-dihydro-[3,4′-bipyridine]-1′,2(2′H)-dicarboxylate in THF (7.9 kg, 7.49 mol, Eq: 1.00) was added over 30 min whilst maintaining the reaction temperature between 20-26° C. After 5 min at 20° C., the reaction mixture was cooled to 0-5° C. (IPC by HPLC). Acetone (658 g, 832 mL, 11.3 mol, Eq: 1.51) was added to the resulting suspension, keeping the temperature between 1 and 4° C. (excess Grignard quench). The suspension was filtered and he filter cake was washed in portions with tetrahydrofuran (in total: 11 kg, 12.5 L). The residue was dried at 40° C. under reduced pressure yielding 2.5 kg of the title product which was introduced in the next step without further purification. tert-Butyl rac-(3'S,5R)-3′-bromo-7,7-dimethyl-spiro[furo[3,4-b]pyridine-5,4′-piperidine]-1′-carboxylate

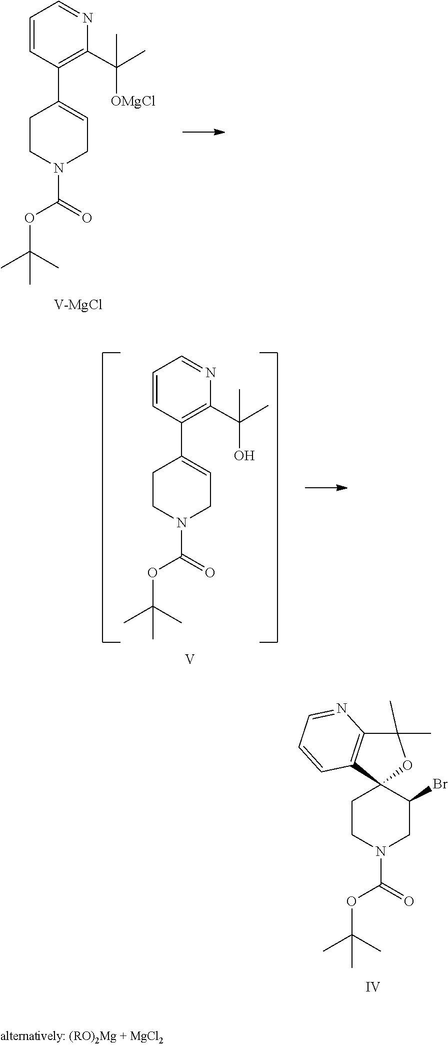

Magnesium 2-(1′-(tert-butoxycarbonyl)-1′,2′,3′,6′-tetrahydro-[3,4′-bipyridin]-2-yl)propan-2-olate chloride (100 g, 265 mmol, Eq: 1.00) was suspended in MTBE (518 g, 700 mL). A 10% aqueous ammonium chloride solution (800 g, 800 mL) was added over 10 min creating a clear biphasic system. The organic phase was separated and washed with deionized water (800 g, 800 mL), concentrated to dryness at 60° C. under reduced pressure rendering 74.3 g of the tertiary alcohol intermediate V.

The crude alcohol V was re-dissolved in acetonitrile (393 g, 500 mL). About 100 mL solvent was distilled at 60° C. under reduced pressure (final volume ca 420 mL). A solution of N-bromosuccinimide (43.6 g, 245 mmol, Eq: 1.05) in acetonitrile (275 g, 350 mL) was added over 1 h at RT. The feed line was washed with acetonitrile (50 mL). After 10 min at RT (IPC by HPLC), a solution of ascorbic acid (6.2 g, 35.2 mmol, Eq: 0.151) in water (90 g, 90 mL) was added in portion at RT (resulting pH 2-3). After another 10 min, a solution of sodium bicarbonate (7 g, 83.3 mmol, Eq: 0.357) in water (110 g, 110 mL) was added over 10 min (resulting pH 7-8, light suspension). About 780 mL of solvent were distilled at 60° C. under reduced pressure. Ethanol (213 g, 270 mL) and water (200 g, 200 mL) were added at 50° C. followed by the addition of seed crystals. The suspension was cooled to RT (0.5° C./min cooling rate). Water (400 g, 400 mL) was added over 1 h. The suspension was stirred at RT overnight and filtered. The filter cake was washed with a 4:1 mixture of a water/ethanol solution (440 mL) and dried at 50° C. under reduced pressure affording 81 g of the title product as white crystals.

### tert-Butyl 7,7-dimethylspiro[furo[3,4-b]pyridine-5,4′-piperidine]-1′-carboxylate

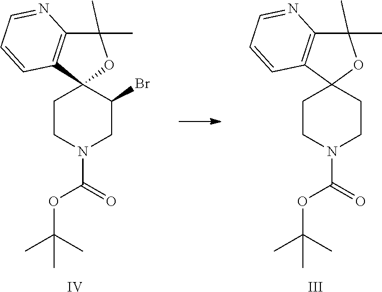

Under an argon atmosphere, tert-butyl rac-(3'S,5R)-3′-bromo-7,7-dimethyl-spiro[furo[3,4-b]pyridine-5,4′-piperidine]-1′-carboxylate (80 g, 201 mmol, Eq: 1.00) and 10% Pd/C (8 g) were charged in an autoclave. Methanol (632 g, 800 mL) and triethylamine (30.6 g, 42 mL, 302 mmol, Eq: 1.50) were added. The vessel was sealed and the mixture stirred for one hour at 25° C. Agitation was stopped and the atmosphere exchanged to hydrogen and adjusted to 4 bar. The mixture was then stirred for 16 h at 25° C. The atmosphere was exchanged to argon and the reaction mixture filtered. The filter was rinsed with methanol (79 g, 100 mL) and the filtrate concentrated under reduced pressure to a volume of 135 mL. The white suspension obtained was taken up in ethyl acetate (270 g, 300 mL) and the solution concentrated to a volume of 135 mL. Ethyl acetate (720 g, 800 mL) and water (320 g, 320 mL) were added under agitation. After separation of the aqueous layer, the organic layer was washed with a solution of sodium bicarbonate (10.2 g, 121 mmol, Eq.: 0.60) in water (200 g, 200 mL) and with water (200 g, 200 mL). The organic layer was concentrated at ambient pressure to a volume of 150 mL. n-Heptane (465 g, 680 mL) was added and a solvent exchange performed at ambient pressure with n-heptane (479 g, 700 mL). The solution was seeded at 80° C., cooled to room temperature and stirred overnight. The product was filtered, washed with n-heptane (164 g, 240 mL) and dried at 50° C./<10 mbar yielding 51.2 g of title compound as a white solid.

### 7,7-Dimethylspiro[furo[3,4-b]pyridine-5,4′-piperidine] dihydrochloride

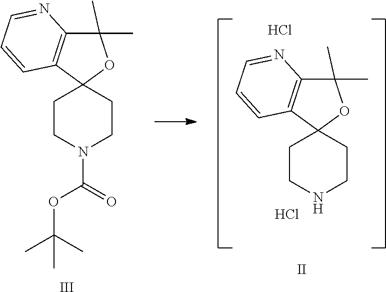

1-Propanol (81.6 g, 102 mL) was charged in a reactor, the temperature set to 0-5° C. and acetylchloride (18.6 g, 16.9 mL, 237 mmol, Eq: 2.1) was added dropwise at this temperature by means of an addition funnel. The addition funnel was rinsed with 1-propanol (28.8 g, 36 mL). The HCl solution in 1-propanol obtained was stirred for one hour at 0-5° C. whilst, separately, tert-butyl 7,7-dimethylspiro[furo[3,4-b]pyridine-5,4′-piperidine]-1′-carboxylate (36.0 g, 113 mmol, Eq: 1.00) in 1-propanol (180 g, 225 mL) was charged in a reactor and heated to 60° C. Maintaining a T₁ of 55-60° C. the prepared HCl solution was added through an addition funnel and the addition funnel was rinsed with 1-propanol (24 g, 30 mL). The reaction mixture was stirred overnight at 60° C., providing the title compound as a light yellow solution or white suspension.

### 2,2-Dimethyl-N-(4′-oxospiro[indane-2,5′-oxazole]-2′-yl)propanamide

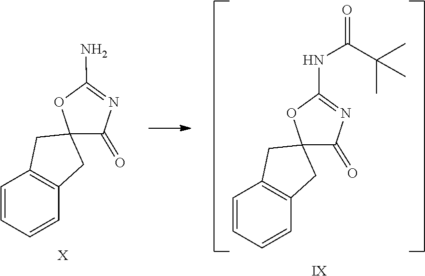

2′-Aminospiro[indane-2,5′-oxazole]-4′-one (22.9 g, 113 mmol, Eq: 1.00) was charged in a reactor and acetonitrile (106 g, 135 mL) was added. To the suspension, pyridine (11.7 g, 12 mL, 148 mmol, Eq: 1.31) was added via an addition funnel and the addition funnel was rinsed with acetonitrile (9.4 g, 12 mL). The suspension was heated to 60-65° C. and pivaloyl chloride (14.2 g, 14.5 mL, 118 mmol, Eq: 1.04) was added using an addition funnel and the addition funnel was rinsed with acetonitrile (10.4 g, 13.2 mL). The reaction mixture was stirred for 4-5 hours at 60° C., generating the title compound as a beige suspension.

### 2′-(7,7-Dimethylspiro[furo[3,4-b]pyridine-5,4′-piperidine]-1′-yl)spiro[indane-2,5′-oxazole]-4′-one

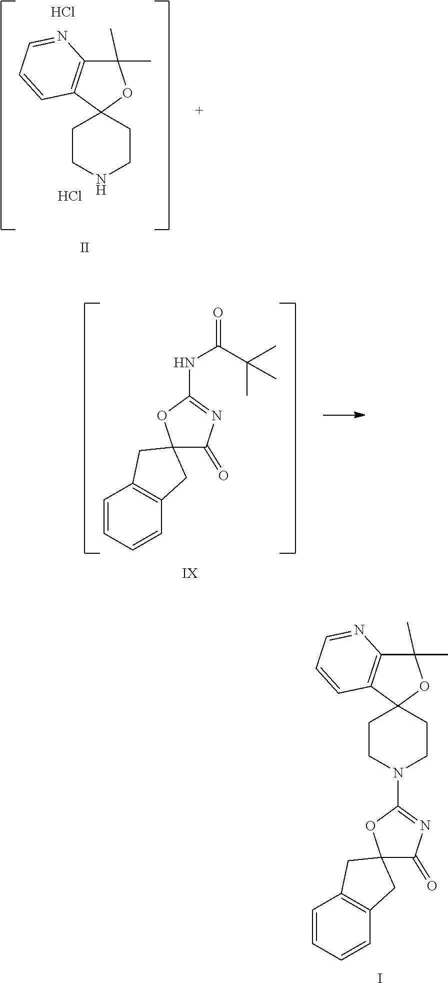

To the previously prepared 2,2-dimethyl-N-(4′-oxospiro[indane-2,5′-oxazole]-2′-yl)propanamide suspension in acetonitrile (reactor 1) heated to 55-60° C. was added N,N-diisopropylethylamine (44.0 g, 59.4 mL, 340 mmol, Eq: 3.01) using an addition funnel which was rinsed thereafter with acetonitrile (10.3 g, 13.2 mL). To the previously prepared 7,7-dimethylspiro[furo[3,4-b]pyridine-5,4′-piperidine]dihydrochloride solution or suspension in 1-propanol (reactor 2) heated to 55-60° C. was added N,N-diisopropylethylamine (14.6 g, 19.8 mL, Eq: 1.00) using an addition funnel which was rinsed thereafter with 1-propanol (8 g, 10 mL). The content of reactor 2 was added over 30 min to the suspension in reactor 1 held at 50-60° C. Reactor 2 was rinsed with 1-propanol (31.2 g, 39 mL). The reaction mixture was stirred for 16 h at 55° C. The light yellow solution was cooled to 30° C. and filtered through an active charcoal cartridge. The equipment was rinsed with 1-propanol (80 g, 100 mL). The solution was concentrated under reduced pressure to 480 mL, water (250 g, 250 mL) was added at 60-80° C. and the mixture then concentrated under reduced pressure to 480 mL. Additional water (300 g, 300 mL) was added over 30 min whilst maintaining T₁ at 65-80° C. The suspension was stirred for one hour and n-heptane (82 g, 120 mL) added over 15 min at 75° C. The suspension was stirred at 75° C. for one hour, cooled over 6 h to 20° C. and stirred overnight (ca. 16 h) at this temperature. The suspension was filtered and the residue was rinsed with a mixture of 1-propanol (28 g, 35 mL) and water (112 g, 112 mL) and then with n-heptane (43.1 g, 63 mL). The product was dried at 60° C./<10 mbar rendering 40.5 g of the title compound as white crystals.

### 2′-(7,7-Dimethylspiro[furo[3,4-b]pyridine-5,4′-piperidine]-1′-yl)spiro[indane-2,5′-oxazole]-4′-one, recrystallized

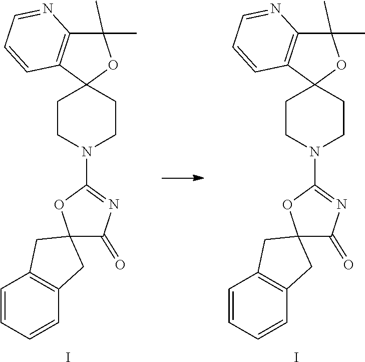

### Recrystallization in 1-Propanol:

2′-(7,7-Dimethylspiro[furo[3,4-b]pyridine-5,4′-piperidine]-1′-yl)spiro[indane-2,5′-oxazole]-4′-one (40.0 g; 99.1 mmol, Eq: 1.00) was charged in a reactor. 1-Propanol (480 g, 598 mL) was added and the suspension heated to 100° C. The hot solution was filtered, the reactor was rinsed with hot (>80° C.) 1-propanol (50 g, 62 mL) and the total volume reduced to 480 mL by distillation at 80-95° C. under reduced pressure. The mixture was heated to 95° C. until a clear solution was obtained. The solution was cooled to 73° C. and seeded. The suspension ensuing was stirred for 1-2 h at 73° C. and temperature was then set to 65° C. The mixture was stirred overnight (ca. 16 h) at 65° C., cooled to 20° C. over 6 h and stirred another ca. 16 h at 20° C. The product was filtered and the residue was washed with 1-propanol (40 g, 50 mL) then dried at 55° C./<10 mbar affording 34.8 g of the title compound as white crystals.

### Recrystallization in Water/1-Propanol:

2′-(7,7-Dimethylspiro[furo[3,4-b]pyridine-5,4′-piperidine]-1′-yl)spiro[indane-2,5′-oxazole]-4′-one (50.0 g; 124 mmol, Eq: 1.00) was charged in a reactor. 1-propanol (600 g, 747 mL) was added and the suspension heated to 100° C. The hot solution was filtered, the reactor rinsed with hot (>80° C.) 1-propanol (50 g, 62 mL) and the total volume reduced to 325 mL by distillation at 7-95° C. under reduced pressure. Water (100 g, 100 mL) was added and the mixture was heated to 85° C. until a clear solution was obtained. The solution was cooled to 73° C. and seeded. The suspension was stirred for 1-2 h at 73° C. and water (700 g, 700 mL) was added over 1 h at the same temperature. After setting the temperature to 65° C., the suspension was stirred overnight (ca. 16 h) at 65° C., cooled to 20° C. over 6 h and stirred another ca. 16 h at 20° C. The product was filtered and the residue was washed with a mixture of 1-propanol (15 g, 19 mL) and water (60 g, 60 mL) then dried under vacuum at 60° C./<10 mbar delivering 48.3 g of the title compound as white crystals.

### REFERENCES

¹ J Neuroendocrinol 2010, 22(5): 362-72.
² Brain Res. 1983, 263(1):105-12.
³ Brain Res Bull. 1981, (2):125-30.
⁴ Science 1984, 223: 833-5.
⁵ Science, 2013, 342: 85-90.
⁶ Heterocycles 1999, 51: 2589
⁷ Synthesis, 2001 (7):1098

1. A crystalline form of a compound of formula I

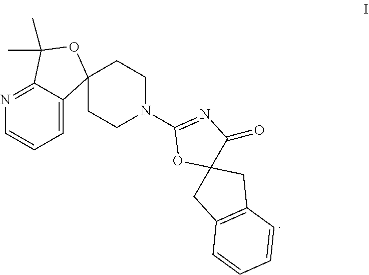 [SMILES: CC1(C)OC2(CCN(C3=NC(=O)C4(Cc5ccccc5C4)O3)CC2)c2cccnc21]

2. The crystalline form of the compound of formula I according to claim 1, wherein the crystalline form is Form A, characterized by a X-ray powder diffraction pattern having the characteristic peaks expressed in values of degrees 2-theta at approximately (±20 degree 2-theta)
degree 2-theta
9.25
9.77
11.13
12.85
13.32
14.48
14.86
17.00
17.37
17.87
18.55
19.60
21.15
21.43
21.63
22.35
22.95
23.37
24.26
24.48

3. The crystalline Form A according to claim 2, characterized by the X-ray powder diffraction pattern as shown in FIG. 1.

4. The crystalline form of the compound of formula I according to claim 1, wherein the crystalline form is Form B, characterized by a X-ray powder diffraction pattern having the characteristic peaks expressed in values of degrees 2-theta at approximately (±20 degree 2-theta)
degree 2-theta
6.66
9.26
9.77
11.12
12.84
13.11
13.34
14.33
14.64
17.01
17.20
17.60
17.88
18.20
18.58
19.61
20.07
21.46
22.33
22.76
23.24
24.06
24.42
24.77
25.84
26.87
28.88
30.91
32.36
34.49
36.21
37.64
39.82

5. The crystalline Form B according to claim 4, characterized by the X-ray powder diffraction pattern as shown in FIG. 4.

6. A process to synthesize a compound of formula I of claim 1, 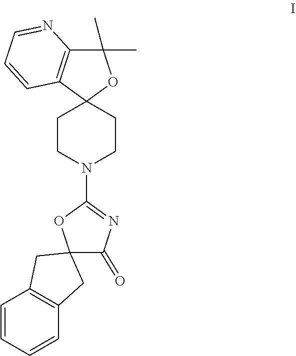 [SMILES: CC1(C)OC2(CCN(C3=NC(=O)C4(Cc5ccccc5C4)O3)CC2)c2cccnc21]
comprising reacting a compound of formula VIII to a compound of formula VII

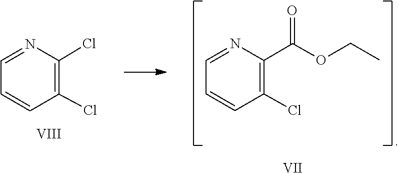

7. The process according to claim 6, wherein the compound of formula VIII is treated in the presence of palladium catalyst, ethanol and an organic base under pressure of carbon monoxide atmosphere at 100±5° C.

8. The process according to claim 7, wherein the compound of formula VIII is treated in the presence of PdCl₂(dppp), ethanol and triethylamine under 60-100 bar pressure of carbon monoxide atmosphere at 100±5° C. and a substrate/catalytic loading greater than 100 and up to 1000.

9. The process according to claim 8, wherein the process further comprises reacting a compound of formula VII with a compound of XV in the presence of a palladium acetate with triphenylphosphine or 4-(di-tert-butylphosphanyl)-N,N-dimethylaniline to a compound of formula VI

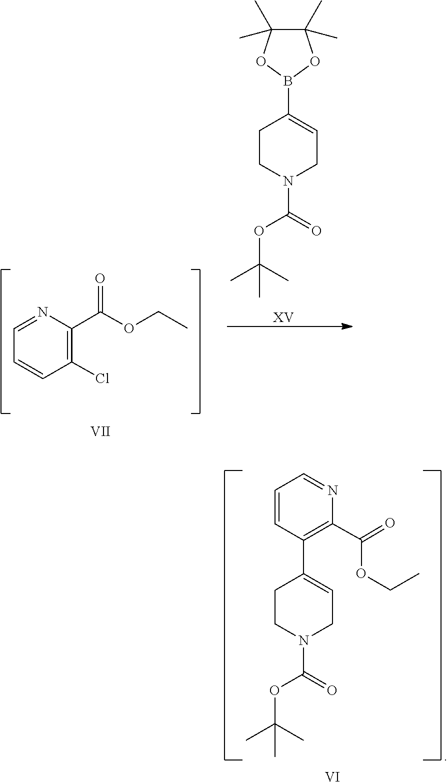

10. The process according to claim 9, wherein the reaction takes place in the presence of a palladium acetate with 4-(di-tert-butylphosphanyl)-N,N-dimethylaniline and tetrabutylammonium bromide at 110° C. in tert-amyl alcohol as solvent.

11. A process to synthesize a compound of formula I according to claim 1, comprising reacting a compound of formula IX with a compound of formula II

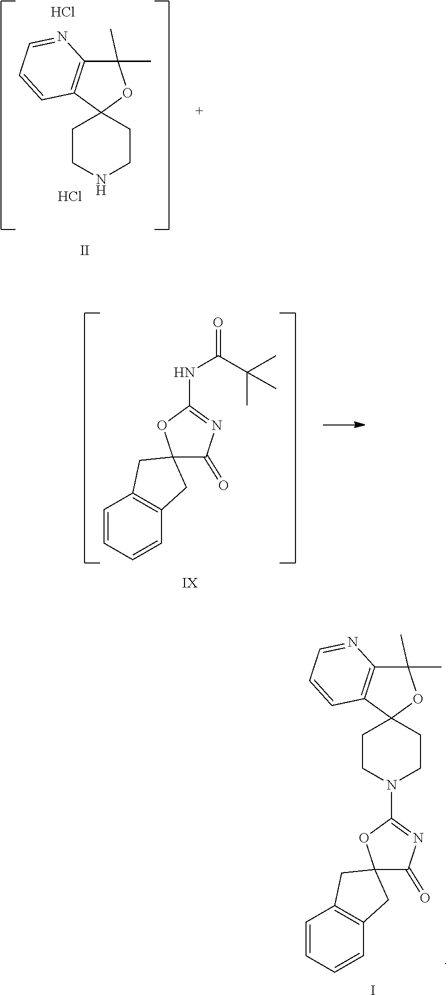

12. The process according to claim 11, further comprising reacting a compound of formula X to a compound of formula IX:

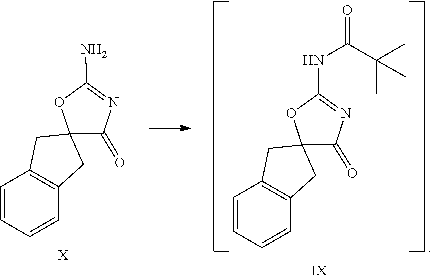

13. The process according to claim 12 further comprising reacting a compound of formula XIV to a compound of formula X or a pharmaceutically acceptable salt thereof via the following steps:

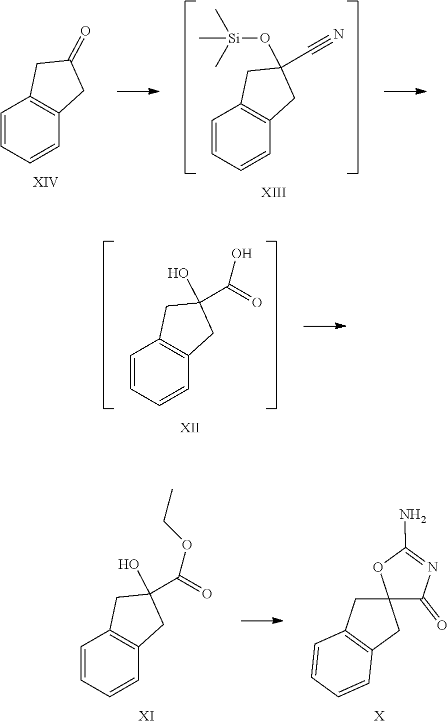

14. The process according to claim 13 further comprising the following steps: [chemical structure]
